(19) United States
(12) Patent Application Publication
(10) Pub. No.: US 2013/0317411 A1
(43) Pub. Date: Nov. 28, 2013

(54) Device for Treatment of Glaucoma

(71) Applicant: Ghansham Das AGARWAL, Kolkata (IN)

(72) Inventor: Ghansham Das AGARWAL, Kolkata (IN)

(21) Appl. No.: 13/478,923

(22) Filed: May 23, 2012

(51) Int. Cl. A61F 9/00

(52) U.S. Cl. 604/8

(57) ABSTRACT

This invention relates to a device for treatment of glaucoma comprising of a circular patch with a plurality of holes wherein the patch is made of biocompatible material. The patch is optionally provided with a projected lip.

### FIELD OF THE INVENTION

This invention relates to a device for treatment of glaucoma.

### BACKGROUND OF INVENTION

Glaucoma is a commonly known eye related disease, in which the pressure of anterior chamber of eye increases, which may lead to blindness if left untreated.

A number of treatments for glaucoma are known to have been provided. They include drops and medications for milder forms and surgical treatment in the form of “trabeculectomy” or filtering surgery and glaucoma shunt.

In the case of a trabeculectomy, an initial pocket is created under the conjunctiva and Tenon's capsule. A flap of partial thickness with its base at corneoscleral junction is provided in the sclera. There is also a provision of window opening under the flap with a punch to remove a portion of sclera, Schlemm's canal and trabecular meshwork so as to enter the anterior chamber of the eye.

The result of this operation is that the scleral flap either does not heal or heals very poorly. Hence, the track thus created allows anterior chamber fluid to come out and get absorbed under the conjunctiva. However, it is associated with a drawback that, due to fibroblast proliferation, the conjunctival wound heals, but leads to failure with time.

To overcome the above, antimetabolites such as mitomycin C or 5 fluorouracil are used. The anticancer drugs prevent healing/fibroblast proliferation at the site. The problem due to poor healing complications is more common. However, it is still the most commonly adopted procedure.

Alternatively, a collagen sponge is placed on top of the scleral flap, which gets absorbed with time. During the healing phase, the fibroblast network thus formed becomes loose, which allows movement and absorption of fluid.

Therefore, a novel device for treatment of glaucoma is required to be developed to further improve and simplify the procedure.

### SUMMARY OF THE INVENTION

The primary object of this invention is to propose a device for the treatment of glaucoma which overcomes the disadvantages associated with the prior art.

Another object of this invention is to propose a device for the treatment of glaucoma to avoid the use of an animal protein (collagen) for the treatment of glaucoma.

Another object of this invention is to propose a device for the treatment of glaucoma to avoid the use of an antimetabolite (anticancer drugs) thereby reducing the complication rates.

Further object of this invention is to propose a device for the treatment of glaucoma to improve results of filtration operation for glaucoma surgery.

Yet another object of this invention is to propose a device for the treatment of glaucoma which is simple, effective, and cost effective.

This invention is a device for treatment of glaucoma comprising of a circular patch with a plurality of holes wherein the patch is made of biocompatible material.

### BRIEF DESCRIPTION OF THE DRAWINGS

Further objects and advantages of this invention will be more apparent from the ensuing description when read in conjunction with the accompanying drawings, and wherein:

FIG. 1 is an exemplary embodiment of the present invention with lip.

FIG. 2 is another exemplary embodiment of the present invention without lip.

### DETAILED DESCRIPTION OF THE INVENTION

The present invention is directed to a device for the treatment of glaucoma. The device comprises a patch of inert material optionally provided with a projected lip. The patch is very thin along with a plurality of holes. Said patch is made of a biologically inert material.

The diameter and thickness of the patch may vary from 6 mm to 16 mm and 0.5 mm to 1.5 mm, respectively. Examples of the materials include medical grade silicone elastomer, a tetrafluoro polyethylene PTFE sheet or a fibrous sheet of same material or other biocompatible materials.

This patch is positioned on top of the scleral flap to cover the distal part of the sclera flap.

In another embodiment, the lip is inserted under the scleral flap and the main patch is kept under the conjunctiva over the sclera. This maintains a passage for fluid from the anterior chamber to under the conjunctiva. Thus, the filtering bleb and passage are maintained without the occurrence of undesirable healing and obliteration of the passage and bleb. The length and width of said lip is 3 mm to 10 mm and 2 mm to 6 mm, respectively. The range for the diameter of the holes in the patch is 0.1 mm to 2 mm.

The instant invention is provided to prevent the healing of the conjunctiva with the sclera. Further, these patches maintain a space/pocket between the two structures.

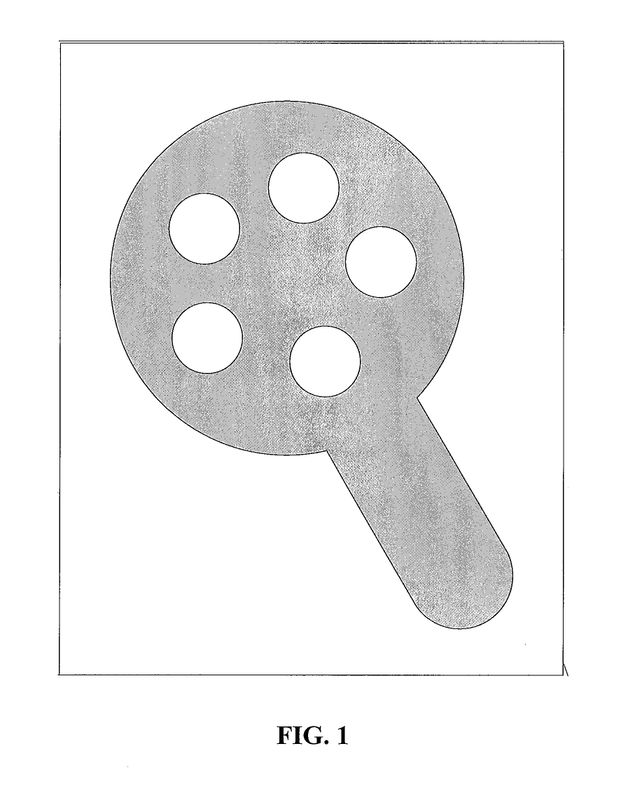

A typical example is a 0.7 mm thick patch with a 10 mm diameter and a 5 mm long with a 3 mm wide lip made of silicone elastomer or PTFE. Also, five holes of 1.5 mm diameter are provided as shown in FIG. 1.

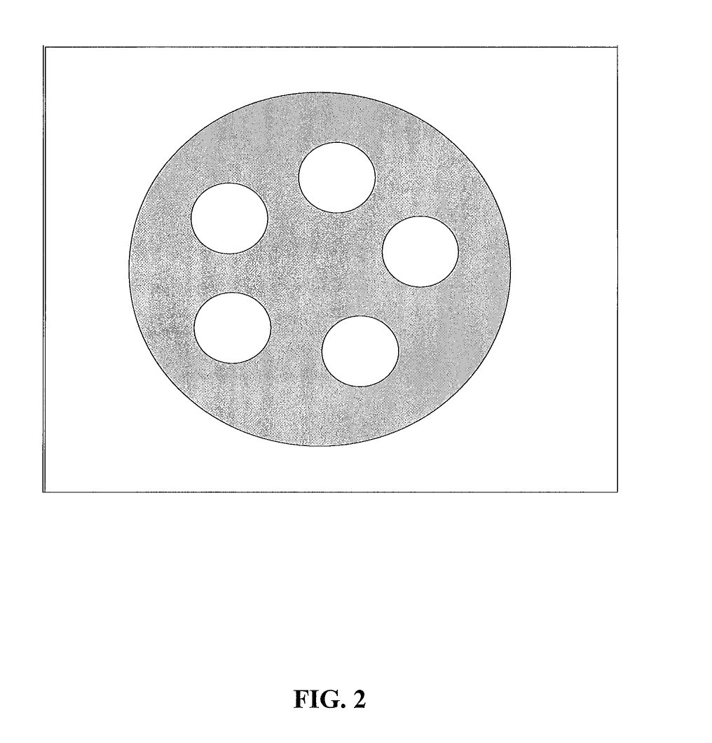

In another embodiment, there is a round patch with a plurality of holes without any lips, as indicated in FIG. 2.

The above-mentioned embodiments may be taken as examples without restricting the scope of the invention to the same.

It is to be noted that the present invention is susceptible to modifications, adaptations, and changes by those skilled in the art. Such variant embodiments employing the concepts and features of this invention are intended to be within the scope of the present invention, which is further set forth under the following claims.

The invention claimed is:

1. A device for treating glaucoma comprising of a circular patch with a plurality of holes wherein the patch is made of a biocompatible material.

2. The device for treating glaucoma as claimed in claim 1, wherein the biocompatible material is selected from the group consisting of a medical grade silicone elastomer, a tetrafluoro polyethylene PTFE sheet, a fibrous sheet and other biocompatible materials.

3. The device for treating glaucoma as claimed in claim 1, wherein the patch has a diameter of 6 mm to 16 mm and a thickness of 0.5 mm to 1.5 mm.

4. The device for treating glaucoma as claimed in claim 1, wherein the patch comprises a projected lip.

5. The device for treating glaucoma as claimed in claim 4, wherein the lip has a length of 3 mm to 10 mm and a width of 2 mm to 6 mm.
